(19) United States
(12) Patent Application Publication
(10) Pub. No.: US 2010/0311989 A1
(43) Pub. Date: Dec. 9, 2010

(54) PROCESS FOR THE PREPARATION OF RENIN INHIBITORS

(76) Inventors: Marlys Hammond, King of Prussia, PA (US); Patrick Stoy, King of Prussia, PA (US); Scott K. Thompson, King of Prussia, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/678,007

(22) PCT Filed: Sep. 17, 2008
(86) PCT No.: PCT/US08/10810

§ 371 Date: Jul. 12, 2010

Related U.S. Application Data
(60) Provisional application No. 60/972,983, filed on Sep. 17, 2007.
(60) Provisional application No. 61/075,815, filed on Jun. 26, 2008.

(51) Int. Cl. C07D 309/04; C07C 33/42

(52) U.S. Cl. 549/426; 568/849

(57) ABSTRACT

Disclosed is a process for the preparation of tetrahydropyran-di-amine represented by Structural Formula (I): wherein R¹ is H or alkyl and E is H or an amine protecting group.

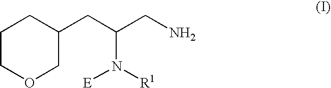

### RELATED APPLICATIONS

This application claims the benefit of U.S. Provisional Application No. 60/972,983, filed on Sep. 17, 2007, and U.S. Provisional Application No. 61/075,815 filed on Jun. 26, 2008. The entire teachings of the above applications are incorporated herein by reference.

### BACKGROUND OF THE INVENTION

Aspartic proteases, including renin, β-secretase (BACE), HIV protease, HTLV protease and plasmepsins I and II, are implicated in a number of disease states. In hypertension, elevated levels of angiotensin I, the product of renin catalyzed cleavage of angiotensinogen are present. Elevated levels of β amyloid, the product of BACE activity on amyloid precursor protein, are widely believed to be responsible for the amyloid plaques present in the brains of Alzheimer's disease patients. The viruses HIV and HTLV depend on their respective aspartic proteases for viral maturation. Plasmodium falciparum uses plasmepsins I and II to degrade hemoglobin.

In the renin-angiotensin-aldosterone system (RAAS), the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension,” in Berkenhager W. H., Reid J. L. (eds): Hypertension, Amsterdam, Elsevier Science Publishing Co, 1996, 489-519; Weber M. A., Am. J. Hypertens., 1992, 5, 247S). Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. Renin inhibitors are not only expected to be superior to ACE inhibitors and AT₁ blockers with regard to safety, but more importantly also with regard to their efficacy in blocking the RAAS.

Recently, non-peptide renin inhibitors were described which show high in vitro activity (Oefner C. et al., Chem. Biol., 1999, 6, 127; Maerki H. P. et al., Il Farmaco, 2001, 56, 21 and International Patent Application Publication No. WO 97/09311). Other non-peptide renin inhibitors have been described in International Patent Application Nos. PCT/US2005/03620 (WO2006/042150), PCT/US2007/008520, and PCT/US2006/043920 (WO2007/070201) and U.S. Provisional Patent Application Nos. 60/845,331 and 60/845,291), the disclosures of each of which are incorporated herein by reference. An example of such aspartic protease/renin inhibitors is a compound represented by Formula (A):

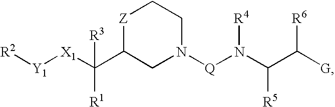

wherein the substituents: R¹, R², R³, R⁴, R⁵, R⁶, X₁, Y₁, Z, Q and G are as defined in PCT/US2006/043920 (WO2007/070201). Another example of an aspartic protease/renin inhibitor is a compound represented by Formula (A-1):

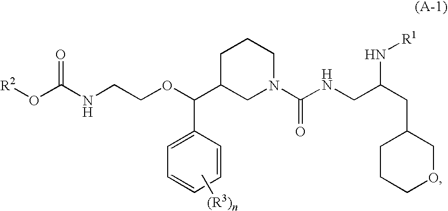

and more specifically a compound represented by Formula (A-2):

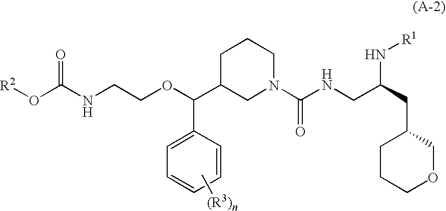

or a pharmaceutically acceptable salt thereof, wherein: R¹ is H or alkyl; R² is alkyl, cycloalkyl or cycloalkylalkyl; R³ is F, Cl, Br, cyano, nitro, alkyl, haloalkyl, alkoxy, haloalkoxy, or alkanesulfonyl; and n is 0, 1, 2, or 3.

The process of forming an aspartic acid protease inhibitor, e.g., represented by Formula (A-1) or (A-2), above, is exemplified in the following scheme:

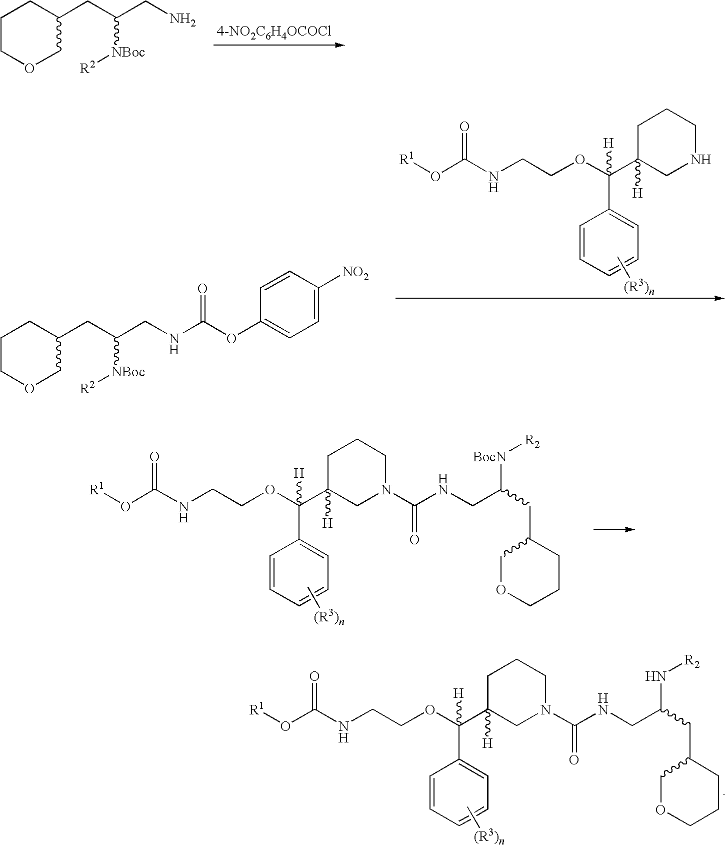

Specific conditions for carrying out the reaction scheme shown above are provided in PCT/US2006/043920 (WO2007/070201), the entire teachings of which are incorporated herein by reference.

Significant quantities of the pure aspartic protease/renin inhibitor are required in the drug development process, e.g., for in vitro and in vivo testing, as formulated and/or un-formulated drug substance. Accordingly, it would be useful to develop efficient processes for the large-scale preparation of such aspartic protease/renin inhibitor compounds and the intermediates used therein.

### SUMMARY OF THE INVENTION

This invention is directed to a process for the preparation of a tetrahydropyran-di-amine represented by Structural Formula (I):

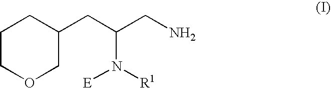

wherein R¹ is H or (C₁-C₆)alkyl and E is H or an amine protecting group, wherein the process comprises the steps of:

1) converting a chloro-pentenol having the formula:

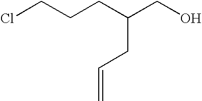

into a propenyl tetrahydropyran having the formula:

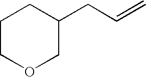

2) converting the propenyl tetrahydropyran into a tetrahydropyran ethylidene methanamine having the formula:

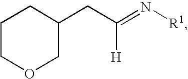

and

3) converting the tetrahydropyran ethylidene methanamine into a cyano-tetrahydropyran-amine having the formula:

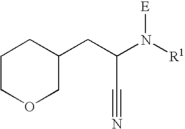

4) converting the tetrahydropyran ethylidene methanamine into the tetrahydropyran-di-amine.

### DETAILED DESCRIPTION OF THE INVENTION

In another embodiment, this invention is directed to a process for the preparation of a tetrahydropyran-di-amine represented by Structural Formulas (Ia), (Ib), (Ic), and (Id):

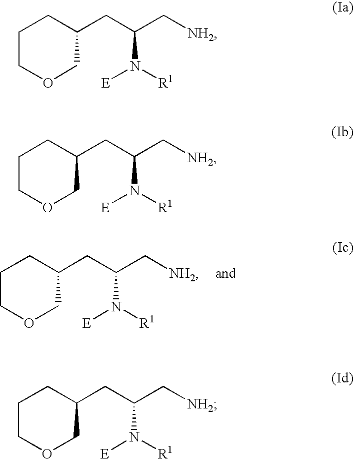

wherein R¹ is H or (C₁-C₆)alkyl and E is H or an amine protecting group.

In the processes of this invention, when E is an amine protecting group, it is understood that E may be any amine protecting group that is compatible with the processes of this invention. Such amine protecting groups are well-known in the art (See T. W. Greene and P. G. M. Wuts “Protective Groups in Organic Synthesis” John Wiley & Sons, Inc., New York 1999). For example, E may be selected from a carbamate, amide, or sulfonamide protecting group. Exemplary amine protecting groups include tert-butoxycarbonyl (Boc), benzyloxycarbonyl (Cbz) and 1-[2-(trimethylsilyl)ethoxycarbonyl] (Teoc).

“Alkyl” means a saturated aliphatic branched or straight-chain hydrocarbon radical. Alkyls commonly have from one to six carbon atoms, typically from one to three carbon atoms. Thus, “(C₁-C₃)alkyl” means a radical having from 1-3 carbon atoms in a linear or branched arrangement. “(C₁-C₃)alkyl” includes methyl, ethyl, propyl and isopropyl.

The starting materials and intermediates used and or prepared in the process of this invention, as well as the product tetrahydropyran di-amines, may exist in various stereoisomeric forms, e.g., as exemplified above in formulas (Ia)-(Id). Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that contain two or more asymmetrically substituted carbon atoms. “R” and “S” represent the configuration of substituents around each one or more chiral carbon atoms. When a chiral center is not defined as R or S and the configuration at the chiral center is not defined by other means, either configuration can be present or a mixture of both configurations can be present.

“Racemate” or “racemic mixture” means a compound of equimolar quantities of two enantiomers, wherein such mixtures exhibit no optical activity; i.e., they do not rotate the plane of polarized light.

“R” and “S” indicate configurations relative to the core molecule. [symbol] and [symbol] represent [symbol] , [symbol] and [symbol] , wherein when [symbol] or [symbol] is used to depict an enantiomer (e.g. [symbol] or [symbol] ), that enantiomer is at least 60%, 70%, 80%, 90%, 99% or 99.9% optically pure.

The processes disclosed herein provide intermediates, as well as the product tetrahydropyran di-amines, as racemic mixtures or as enantiomerically or diastereomerically enriched mixtures. Such enantiomerically or diastereomerically enriched mixtures are at least 60%, 70%, 80%, 90%, 99% or 99.9% optically pure. Purified, individual isomers (enantiomers or diastereomers) may be obtained by resolution from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture using various well known chromatographic methods.

When the stereochemistry of the intermediates, as well as the product tetrahydropyran di-amines, are named or depicted by structure, the named or depicted stereoisomer(s) is (are) at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% optically pure.

Salts, specifically pharmaceutically acceptable salts, of the disclosed intermediates and product tetrahydropyran di-amines may be obtained by reacting the amine compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999, and the entire teaching of which is herein incorporated by reference. Protecting groups may be added and removed using methods well known in the art.

In one embodiment, this invention is directed to a process for the preparation of a tetrahydropyran-di-amine represented by the formula:

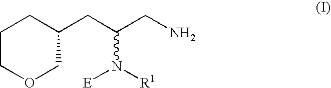

wherein the process comprises the steps of:

1) converting a chloro-pentenol having the formula:

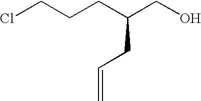

into a propenyl tetrahydropyran having the formula:

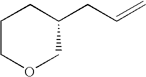

2) converting the propenyl tetrahydropyran into a tetrahydropyran ethylidene methanamine having the formula:

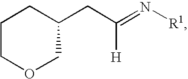

and

3) converting the tetrahydropyran ethylidene methanamine into a cyano-tetrahydropyran-amine having the formula:

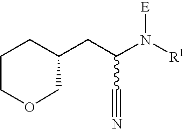

4) converting the tetrahydropyran ethylidene methanamine into the tetrahydropyran-di-amine.

Another embodiment of the invention is the reaction in step 1) above. Another embodiment of the invention is the reaction in step 2) above. Another embodiment of the invention is the reaction in step 3) above. Another embodiment of the invention is the reaction in step 4) above.

Another embodiment, this invention is directed to a process for the preparation of a tetrahydropyran-di-amine represented by the formula:

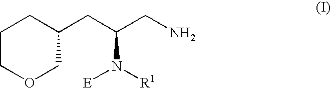

wherein the process comprises the steps of:

1) converting a chloro-pentenol having the formula:

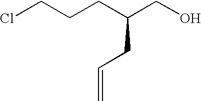

into a propenyl tetrahydropyran having the formula:

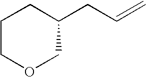

2) converting the propenyl tetrahydropyran into a tetrahydropyran ethylidene methanamine having the formula:

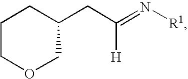

and

3) converting the tetrahydropyran ethylidene methanamine into a cyano-tetrahydropyran-amine having the formula:

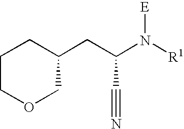

4) converting the tetrahydropyran ethylidene methanamine into the tetrahydropyran-di-amine.

Another embodiment of the invention is the reaction in step 1) above. Another embodiment of the invention is the reaction in step 2) above. Another embodiment of the invention is the reaction in step 3) above. Another embodiment of the invention is the reaction in step 4) above.

In another embodiment, this invention is directed to a process for the preparation of a chloro-pentenol having the formula:

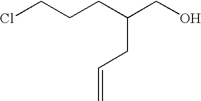

comprising the steps of:

1) treating pseudoephedrine with 5-chloropentanoyl chloride to form a pentanamide having the formula:

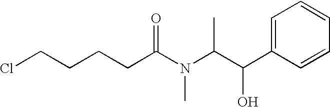

2) converting the pentanamide to a pentenamide having the formula:

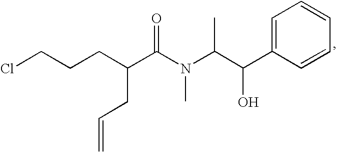

and

3) converting the pentenamide into the chloro-pentenol.

The pseudoephedrine used in the process of this invention may be racemic or may be stereoisomerically pure. For example, (1S,2S)-pseudoephedrine or (1R,2R)-pseudoephedrine may be used to form

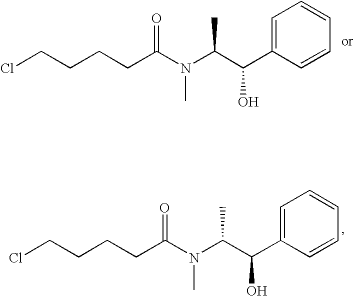

respectively.

Another embodiment of the invention is the reaction in step 1) above. Another embodiment of the invention is the reaction in step 2) above. Another embodiment of the invention is the reaction in step 3) above. Another embodiment of the invention is the reaction in step 4) above.

In the process of this invention, the reaction of the pseudoephedrine with 5-chloropentanoyl chloride is conducted using general amide-forming reactions conditions, for example, by conducting the reaction (e.g., at reduced temperature) in the presence of a mild base, e.g., an amine base such as triethylamine. See: J. Am. Chem. Soc. 1994, 116, 9361. Transformation of the pentanamide into a pentenamide is conducted using general alkylation conditions, e.g., treatment of the amide with a base prior to treatment with an alkylating agent. In the process of this invention, the pentanamide may be first treated with an amide base such as lithium diisopropylamide (LDA) (e.g., formed in situ using diethylamine and n-BuLi), lithium tetramethylpiperdide or lithium dicyclohexylamide and optionally a lithium salt such as LiCl, then, allylbromide. The chloro-pentenol is formed from the pentenamide by reduction of the pseudoephedrine amide with a reagent suitable for converting an amide to an alcohol. See: Tetrahedron Lett. 1996, 37, 3623, the entire teachings of which are incorporated herein by reference. In another embodiment, this invention is directed to a process for the preparation of an R-chloro-pentenol having the formula:

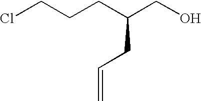

comprising the steps of:

1) treating S,S-pseudoephedrine with 5-chloropentanoyl chloride to form an R,S,S-pentanamide having the formula:

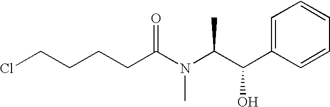

2) converting the R,S,S-pentanamide to an R,S,S-pentenamide having the formula:

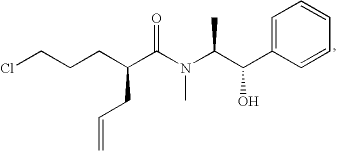

and

3) converting the R,S,S-pentenamide into the R-chloro-pentenol.

Another embodiment of the invention is the reaction in step 1) above. Another embodiment of the invention is the reaction in step 2) above. Another embodiment of the invention is the reaction in step 3) above. Another embodiment of the invention is the reaction in step 4) above.

In another embodiment, this invention is directed to a process for the preparation of the tetrahydropyran ethylidene methanamine having the formula:

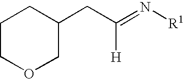

comprising the steps of:

1) converting a chloro-pentenol having the formula:

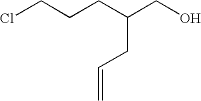

into a propenyl tetrahydropyran having the formula:

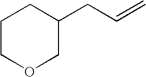

2) converting the propenyl tetrahydropyran into a tetrahydropyran acetaldehyde having the formula:

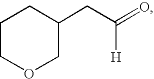

and

3) converting the tetrahydropyran acetaldehyde into the tetrahydropyran ethylidene methanamine.

Another embodiment of the invention is the reaction in step 1) above. Another embodiment of the invention is the reaction in step 2) above. Another embodiment of the invention is the reaction in step 3) above.

Another embodiment, this invention is directed to a process for the preparation of an R-tetrahydropyran ethylidene methanamine having the formula:

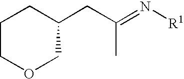

comprising the steps of:

1) converting an R-chloro-pentenol having the formula:

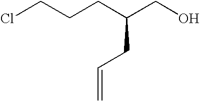

into an R-propenyl tetrahydropyran having the formula:

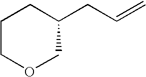

2) converting the R-propenyl tetrahydropyran into an R-tetrahydropyran acetaldehyde having the formula:

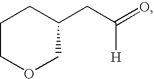

and

3) converting the R-tetrahydropyran acetaldehyde into the R-tetrahydropyran ethylidene methanamine.

Another embodiment of the invention is the reaction in step 1) above. Another embodiment of the invention is the reaction in step 2) above. Another embodiment of the invention is the reaction in step 3) above.

In the process of this invention, the chloropentenol may be converted into the propenyl tetrahydropyran by treatment with a base, for example, a hydride base such as KH, LiH or NaH. The propenyl tetrahydropyran may be converted to the tetrahydropyran acetaldehyde by conventional oxidative methods, for example using RuCl₃ and NaIO₄. Formation of the alkyl-imine, the tetrahydropyran ethylidene methanamine, from the tetrahydropyran acetaldehyde may be accomplished using a desired alkylamine under conventional conditions, for example using methylamine in the presence of molecular sieves or other dehydrating reagent.

As described above the tetrahydropyran ethylidene methanamine is first converted into a cyano-tetrahydropyran-amine, which is subsequently converted into the tetrahydropyran di-amine. Introduction of the cyano moiety is accomplished using 3-{(E)-[((1R,2R)-2-{[({(1S)-1-[(dimethylamino)carbonyl]-2,2-dimethylpropyl}amino)carbonothioyl]amino}cyclohexyl)imino]methyl}-5-(1,1-dimethylethyl)-4-hydroxyphenyl 2,2-dimethylpropanoate and trimethylsilanecarbonitrile, followed by formation of the Boc protecting group using bis(1,1-dimethylethyl) dicarbonate. The last step of this process comprises the reduction of the cyano group to form the methylene-amino moiety of the tetrahydropyran di-amine. This reaction may be conducted using a variety of reducing agents, for example by hydrogenation using a suitable hydrogenation catalyst such as Raney-nickel.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art.

The following abbreviations have the indicated meanings:

| Abbreviation | Meaning |
|---|---|
| Aq | Aqueous |
| Boc | tert-butoxy carbonyl or t-butoxy carbonyl |
| (Boc)₂O | di-tert-butyl dicarbonate |
| Brine | saturated aqueous NaCl |
| Cbz | Benzyloxycarbonyl |
| CbzCl | Benzyl chloroformate |
| CDI | carbonyl diimidazole |
| CH₂Cl₂ | methylene chloride |
| CH₃CN or MeCN | Acetonitrile |
| Cpd | Compound |
| D | Day |
| DAMP | 4,4′-(2-pyridinylmethylene)diphenol acetate |
| DAST | diethylaminosulfur trifluoride |
| DBU | 1,8-diazabicyclo[5.4.0]undec-7-ene |
| DCC | N,N′-dicyclohexylcarbodiimide |
| DCM | Dichloromethane |
| DCU | N,N′-dicyclohexylurea |
| DIAD | diisopropyl azodicarboxylate |
| DiBAlH | Diisobutylaluminum hydride |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| 2,4-DNP | 2,4-dinitrophenylhydrazine |
| DPPA | Diphenylphosphoryl azide |
| EDCI•HCl | 1-[3-(dimethylamino)propyl]-3- |
| | ethylcarbodiimide |
| | hydrochloride |
| Equiv | Equivalents |
| Et | Ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]- |
| | 2,5-pyrrolidinedione |
| h, hr | Hour |
| HOBt | 1-hydroxybenzotriazole |
| HATU | 2-(7-Aza-1H-benzotriazole-1-yl)-1,1,3,3- |
| | tetramethyluronium |
| | hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LiHMDS | lithium hexamethyldisilazane |
| LAB | lithium amidotrihydroborate |
| LAH or LiAlH₄ | lithium aluminum hydride |
| LC-MS | liquid chromatography-mass spectroscopy |
| LHMDS | lithium hexamethyldisilazane |
| Me | Methyl |
| MeCN | Acetonitrile |
| MeOH | Methanol |
| MsCl | Methanesulfonyl chloride |
| Min | Minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMM | N-methylmorpholine |
| NMP | N-methylpyrrolidinone |
| Pd₂(dba)₃ | tris(dibenzylideneacetone)dipalladium(0) |
| PE | petroleum ether |
| Ph | Phenyl |
| PTSA | p-toluene sulfonic acid |
| R-CBS | (R)-CBS-oxazaborolidine |
| Quant | quantitative yield |
| Rt | room temperature |
| Satd | Saturated |
| SOCl₂ | thionyl chloride |
| SPE | solid phase extraction |
| TBDPSCl | tert-butyl diphenyl silyl chloride |
| TBME | tert-butyl methyl ether |
| TBS | t-butyldimethylsilyl |
| TBSCl | t-butyldimethylsilyl chloride |
| TEA | triethylamine or Et₃N |
| TEAF | tetraethylammonium fluoride |
| TEMPO | 2,2,6,6-tetramethyl-1-piperidinyloxy free radical |
| Teoc | 1-[2-(trimethylsilyl)ethoxycarbonyloxy]- |
| Teoc-OSu | 1-[2-(trimethylsilyl)ethoxycarbonyloxy]pyrrolidin- |
| | 2,5-dione |
| TFA | trifluoroacetic acid |
| THF | Tetrahydrofuran |
| Tlc | thin layer chromatography |
| TMS | Trimethylsilyl |
| TMSCl | chlorotrimethylsilane or trimethylsilyl chloride |
| t_{R} | retention time |
| TsOH | p-toluenesulfonic acid |
| TsCl | p-toluenesulfonyl chloride |
| Red-Al | sodium bis(2-methoxyethoxy)aluminum dihydride |

### Example 1

tert-Butyl (S)-1-amino-3-((R)-tetrahydro-2H-pyran-3-yl)propan-2-yl(methyl)carbamate may be prepared by the following procedures:

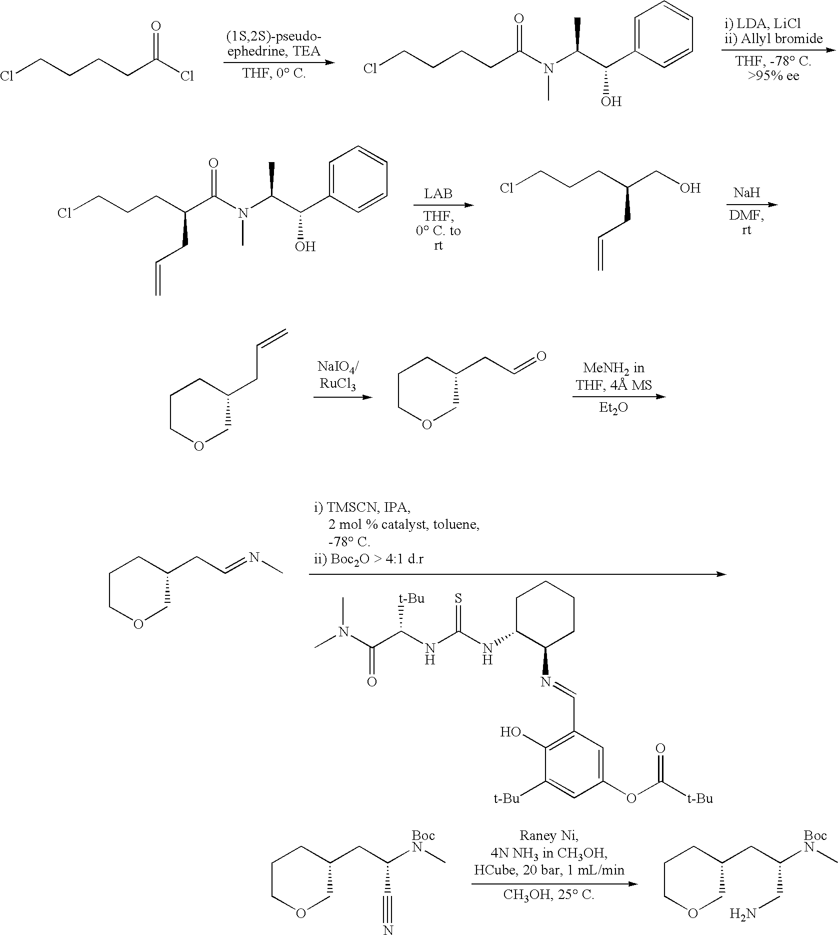

### Step 1. 5-Chloro-N-((1S,2S)-1-hydroxy-1-phenylpropan-2-yl)-N-methylpentanamide

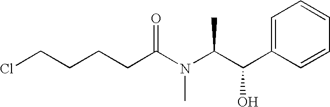

To a magnetically stirred solution of (1S,2S)-pseudoephedrine (60 g, 363.1 mmol) in THF (600 mL) at room temperature was added triethylamine (65.4 mL, 472 mmol) in one portion. The resulting white suspension was cooled to 0° C. A solution of 5-chloropentanoyl chloride (49 mL, 381 mmol) in THF (130 mL) was added dropwise to the mixture over 45 min using an addition funnel. The mixture was then allowed to stir at 0° C. for 30 min. H₂O (40 mL) was added and the resulting mixture was concentrated to ˜10% of the original volume. The resulting solution was partitioned between H₂O/EtOAc and the layers were separated. The aqueous layer was extracted with EtOAc (600 mL). The combined organic layers were washed with saturated aqueous NaHCO₃, brine, dried over MgSO₄, filtered, and concentrated under reduced pressure to furnish the crude product as pale yellow oil. The crude amide was purified by flash chromatography (ISCO; 3×330 g column; CH₂Cl₂ to 5% MeOH/CH₂Cl₂) to provide the product as a clear, viscous oil. The residual MeOH was removed through co-evaporating with toluene (3×100 mL) to provide 5-chloro-N-((1S,2S)-1-hydroxy-1-phenylpropan-2-yl)-N-methylpentanamide (96.2 g, 339 mmol, 93%). LCMS (m/z=266.0)

### Step 2. (R)-2-(3-Chloropropyl)-N-((1S,2S)-1-hydroxy-1-phenylpropan-2-yl)-N-methylpent-4-enamide

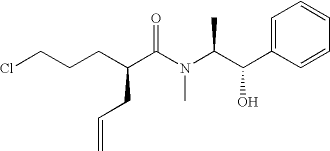

To a magnetically stirred suspension of LiCl (83 g, 1.96 mol) in THF (700 mL) at room temperature was added diisopropylamine (104 mL, 736 mmol) in one portion. nBuLi (2.5M in hexane, 281 mL, 703 mmol) was added dropwise over 30 min using an addition funnel. The light yellow mixture was stirred at −78° C. for 20 min and then was warmed to 0° C. for 15 min. The mixture was then cooled to −78° C. and 5-chloro-N-((1S,2S)-1-hydroxy-1-phenylpropan-2-yl)-N-methylpentanamide (92.8 g, 327 mmol) in THF (330 mL) was added dropwise over 30 min using an addition funnel. The mixture was stirred at −78° C. for 1 h and then was warmed to 0° C. for 25 min. Allyl bromide (41.5 mL, 490 mmol) was then added slowly over 2 min via syringe and then the reaction was warmed to room temperature. The reaction mixture was stirred at room temperature for 50 min and was judged complete by LC/MS analysis. The mixture was cooled to 0° C. and saturated aqueous NaHCO₃ (400 mL) and H₂O (200 mL) were added. EtOAc was added, the phases were separated and the aqueous phase was extracted with EtOAc (1500 mL total). The combined organic layers were washed with 1N HCl (4×150 mL), brine, dried over MgSO₄, filtered, and concentrated under reduced pressure to furnish (R)-2-(3-chloropropyl)-N-((1S,2S)-1-hydroxy-1-phenylpropan-2-yl)-N-methylpent-4-enamide as an orange oil (101.2 g, 312 mmol, 95%). The crude material was carried on without further purification. LC/MS (m/z=306.0).

### Step 3. (R)-2-(3-Chloropropyl)pent-4-en-1-ol

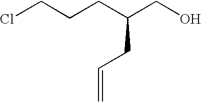

A magnetically stirred solution of diisopropylamine (184 mL, 1.29 mol) in THF (600 mL) was cooled to −78° C. nBuLi (2.5M in hexane, 482 mL, 1.21 mol) was added dropwise over 35 min using an addition funnel. The cloudy mixture was stirred at −78° C. for 15 min and then was warmed to 0° C. for 15 min during which time the solution became clear and light yellow. Borane-ammonia complex (90%, 42 g, 1.24 mol) was added in four equal portions, one minute apart. (Caution: vigorous evolution of gas). The cloudy mixture was warmed to room temperature for 20 min and then was recooled to 0° C. (R)-2-(3-chloropropyl)-N-((1S,2S)-1-hydroxy-1-phenylpropan-2-yl)-N-methylpent-4-enamide (100.2 g, 309 mmol) in THF (300 mL) was added dropwise over 10 min using an addition funnel. The reaction mixture was warmed to room temperature and stirred for 2.5 h. The reaction mixture was then cooled to −10° C. and was quenched with HCl (3M, 1500 mL). The phases were separated and the aqueous phase was extracted with Et₂O (2000 mL total). The combined organic layers were washed with 3N HCl, brine, dried over MgSO₄, filtered, and concentrated under reduced pressure to furnish the crude product as a yellow oil. The crude material was purified by flash chromatography (ISCO; 330 g column; Hexane to 30% EtOAc/Hexane) to provide (R)-2-(3-chloropropyl)pent-4-en-1-ol as a clear, viscous oil (32.6 g, 200 mmol, 65%); ¹H NMR (400 MHz. CDCl₃) δ 5.82 (m, 1H), 5.07 (m, 2H), 3.78 (m, 1H), 3.58 (d, J=8.0 Hz, 2H), 3.54 (t, J=8 Hz, 2H), 2.14 (m, 2H), 1.85 (m, 2H), 1.64 (m, 1H), 1.49 (m, 1H).

### Step 4. (R)-3-Allyl-tetrahydro-2H-pyran

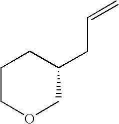

DMF (350 mL) was added to a round bottom flask containing NaH (60% w/w, 15 g, 0.376 mmol) and a magnetic stir bar. The suspension was cooled to 5-10° C. in an ice bath and stirred for 5 min. A solution of (R)-2-(3-chloropropyl)pent-4-en-1-ol (30.6 g, 188 mmol) in DMF (350 mL) was added via addition funnel over 25 min. Caution: Gas evolution and exotherm. The resulting creamy suspension was stirred for 30 min. The reaction mixture was warmed to room temperature and the resulting beige suspension was stirred for 2 h, at which time the reaction was judged to be complete by TLC. The reaction mixture was cooled to 0° C. and quenched by addition of H₂O (250 mL) and HCl (3N, 250 mL). The phases were separated and the aqueous phase was extracted with petroleum ether (4×250 mL). The combined organic layers were washed with H₂O, brine, dried over MgSO₄, filtered, and concentrated under reduced pressure to furnish the crude product as a yellow oil. The crude material was purified by flash chromatography (ISCO; 120 g column; Hexane to 30% EtOAc/Hexane) to provide (R)-3-allyl-tetrahydro-2H-pyran as a clear oil (19.8 g, 157 mmol, 83%); ¹H NMR (400 MHz. CDCl₃) δ 5.72-5.82 (m, 1H), 5.00-5.06 (m, 2H), 3.86-3.91 (m, 2H), 3.37 (m, 1H), 3.08 (t, J=12 Hz, 1H), 1.85-1.98 (m, 3H), 1.59-1.69 (m, 3H), 1.15-1.21 (m, 1H).

### Step 5. (R)-2-(Tetrahydro-2H-pyran-3-yl)acetaldehyde

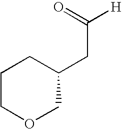

To a magnetically stirred solution of (R)-3-allyl-tetrahydro-2H-pyran (18.7 g, 148 mmol) in acetonitrile (740 mL) at room temperature was added RuCl₃.2H₂O (1.43 g, 5.92 mmol) in one portion. The resulting dark brown solution was stirred at room temperature for 5 min and then NaIO₄ (69 g, 326 mmol) was added in one portion. H₂O was added in small portions (10×8 mL) at 5 min intervals. The reaction mixture was stirred at room temperature for 30 min, at which time the reaction was judged complete by TLC. The reaction mixture was quenched by addition of saturated aqueous Na₂S₂O₃ (250 mL) and H₂O (1000 mL). The phases were separated and the aqueous phase was extracted with Et₂O (4×400 mL). The combined organic layers were washed with H₂O, brine, dried over MgSO₄, filtered, and concentrated under reduced pressure to furnish the crude product as a yellow oil. The crude material was purified by flash chromatography (ISCO; 120 g column; Hexane to 40% EtOAc/Hexane) to provide (R)-2-(tetrahydro-2H-pyran-3-yl)acetaldehyde as a yellow oil (14.3 g, 111 mmol, 60%); ¹H NMR (400 MHz, CDCl₃) δ 9.78 (t, J=2, 1H), 3.84-3.88 (m, 2H), 3.40-3.47 (m, 1H), 3.17 (dd, J=11.2, 8.8 Hz, 1H), 2.31-2.41 (m, 2H), 2.21-2.28 (m, 1H), 1.88-1.93 (m, 1H), 1.61-1.72 (m, 2H), 1.29-1.33 (m, 1H).

### Step 6. (R,E)-N-(2-(Tetrahydro-2H-pyran-3-yl)ethylidene)methanamine

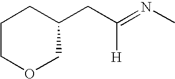

To a magnetically stirred solution of (R)-2-(tetrahydro-2H-pyran-3-yl)acetaldehyde (11 g, 85.8 mmol) in Et₂O (215 mL) at room temperature was added MeNH₂ (2M in THF, 215 mL, 429.2 mmol) and molecular sieves (4 Å, powdered, activated, 21.5 g). The reaction mixture was stirred at room temperature for 1 h. The resulting mixture was then filtered and concentrated under reduced pressure to furnish (R,E)-N-(2-(tetrahydro-2H-pyran-3-yl)ethylidene)methanamine as a yellow oil (11.3 g, 80 mmol, 93%). The crude material was carried on without further purification. ¹H NMR (400 MHz, CDCl₃) δ 7.67 (m, 1H), 3.86-3.91 (m, 2H), 3.36-3.43 (m, 1H), 3.29 (s, 3H), 3.13 (dd, J=11.0, 9.8 Hz, 1H), 1.95-2.14 (m, 2H), 1.86-1.91 (m, 2H), 1.62-1.68 (m, 2H), 1.21-1.30 (m, 1H).

NMR analysis revealed the presence of only a single geometric isomer which was assigned as the E-isomer, based on literature precedent. Non-detectable (not detected by NMR) amounts of the Z-isomer may also have been formed.

### Step 7. tert-Butyl (S)-1-cyano-2-((R)-tetrahydro-2H-pyran-3-yl)ethyl(methyl)-carbamate

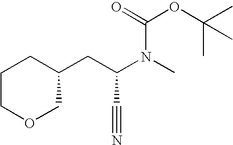

A 2 L, round bottom flask was charged with toluene (400 mL), a magnetic stir bar, (R,E)-N-(2-(tetrahydro-2H-pyran-3-yl)ethylidene)methanamine (11.3 g, 80.1 mmol) and 3-{(E)-[((1R,2R)-2-{[({(1S)-1-[(dimethylamino)carbonyl]-2,2-dimethylpropyl}amino)carbonothioyl]amino}cyclohexyl)imino]methyl}-5-(1,1-dimethylethyl)-4-hydroxyphenyl 2,2-dimethylpropanoate (J. Am. Chem. Soc., 2002, 124, 10012-10014) (0.9 g, 1.6 mmol). The mixture was cooled to −78° C. and trimethylsilanecarbonitrile (21.4 mL, 160.2 mmol) was added dropwise over 15 min using an addition funnel. Isopropyl alcohol (12.3 mL, 160.2 mmol) was then added dropwise over 10 min. The reaction mixture was stirred at −78° C. for 3 h and then was warmed to room temperature and stirred for 1 h. Bis(1,1-dimethylethyl) dicarbonate (35.0 g, 160.2 mmol) was then added and the resulting mixture was stirred at room temperature for 1 h. The reaction was quenched by the addition of saturated aqueous NaHCO₃ (400 mL) and EtOAc (300 mL). The layers were separated and the aqueous layer was washed with EtOAc (100 mL). The combined organic layers were dried over Na₂SO₄, filtered, and concentrated under reduced pressure to give the crude product. The crude material was divided into two parts and each was purified by flash chromatography (ISCO; 120 g column; 0% to 10% EtOAc/Hexane over 30 min, then 10% EtOAc/Hexane 47 min, then 10% to 20% EtOAc/Hexane over 2 min, then 20% EtOAc/Hexane for 11 min). The two purified batches were combined to provide tert-butyl (S)-1-cyano-2-((R)-tetrahydro-2H-pyran-3-yl)ethyl(methyl)carbamate (18.9 g, 70 mmol, 86%) as an orange oil.

(Product is a 4:1 mixture of diastereomers) ¹H NMR (400 MHz, CDCl₃) δ 5.00 (brs, 1H), 3.83-3.90 (m, 2H), 3.42-3.48 (m, 1H), 3.19 (dd, J=11.3, 8.6, 1H), 2.92 (s, 3H), 1.85-1.95 (m, 1H), 1.60-1.82 (m, 5H), 1.50 (s, 9H), 1.28-1.33 (m, 1H).

### Step 8. tert-Butyl (S)-1-amino-3-((R)-tetrahydro-2H-pyran-3-yl)propan-2-yl(methyl)carbamate

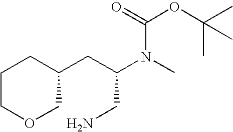

tert-Butyl (S)-1-cyano-2-((R)-tetrahydro-2H-pyran-3-yl)ethyl(methyl)carbamate (397 mg, 4:1 mixture of diastereomers at the alpha-amino stereocenter) was dissolved in a solution of 4M NH₃ in MeOH (15 mL) and passed through a Raney-nickel cartridge (CatCart®, 50 mm) on an in-line hydrogenation apparatus (H-Cube) with the following settings: ambient temperature (14° C.), flow rate 1.0 mL/min, H₂ pressure 30 atm. The solution was recirculated so that the product solution was fed back into the apparatus. After thirty minutes, TLC analysis (1:9 MeOH/CH₂Cl₂, KMnO₄ stain) showed complete conversion of the starting material. After 60 min total reaction time, the solution was evaporated to yield 371 mg (92%) of tert-butyl (S)-1-amino-3-((R)-tetrahydro-2H-pyran-3-yl)propan-2-yl(methyl)carbamate as a clear, rose-colored oil. LC-MS (ELSD) m/e 273.6 (M+H)⁺.

### Example 2

(S)-2-(3-Chloropropyl)pent-4-en-1-ol may be prepared by the following procedures:

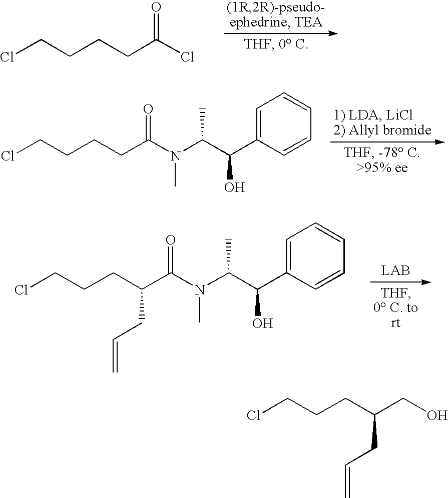

### Step 1. 5-Chloro-N-((1R,2R)-1-hydroxy-1-phenylpropan-2-yl)-N-methylpentanamide

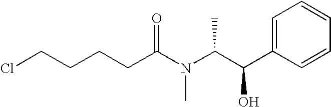

5-Chloro-N-((1R,2R)-1-hydroxy-1-phenylpropan-2-yl)-N-methylpentanamide was prepared from 5-chloropentanoyl chloride (7.8 mL, 60.4 mmol) and (1R,2R)-pseudoephedrine (9.9 g, 60.4 mmol) according to the method described in Example 1, Step 1.

### Step 2. (S)-2-(3-Chloropropyl)-N-((1R,2R)-1-hydroxy-1-phenylpropan-2-yl)-N-methylpent-4-enamide

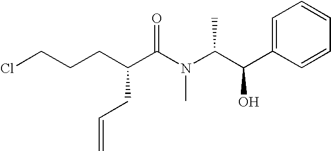

(S)-2-(3-Chloropropyl)-N-((1R,2R)-1-hydroxy-1-phenylpropan-2-yl)-N-methylpent-4-enamide was prepared from 5-chloro-N-((1R,2R)-1-hydroxy-1-phenylpropan-2-yl)-N-methylpentanamide (17.7 g, 60.2 mmol) according to the method described in Example 1, Step 2.

### Step 3. (S)-2-(3-Chloropropyl)pent-4-en-1-ol

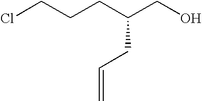

(S)-2-(3-Chloropropyl)pent-4-en-1-ol was prepared from (S)-2-(3-chloropropyl)-N-((1R,2R)-1-hydroxy-1-phenylpropan-2-yl)-N-methylpent-4-enamide (18.2 g, 56.2 mmol) according to the method described in Ex. 1, Step 3.

While this invention has been particularly shown and described with references to specific embodiments thereof, it will be understood by those skilled in the art that various changes in form and details may be made therein without departing from the scope of the invention encompassed by the appended claims.

1. A process for the preparation of a tetrahydropyran-di-amine represented by Structural Formula (I): 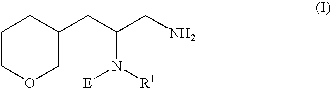
wherein R¹ is H or (C₁-C₆)alkyl and E is H or an amine protecting group, wherein the process comprises the steps of:
1) converting a chloro-pentenol having the formula: 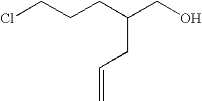
into a propenyl tetrahydropyran having the formula: 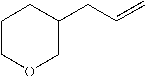
2) converting the propenyl tetrahydropyran into a tetrahydropyran ethylidene methanamine having the formula: 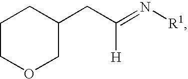
and
3) converting the tetrahydropyran ethylidene methanamine into a cyano-tetrahydropyran-amine having the formula: 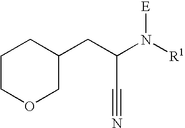
4) converting the tetrahydropyran ethylidene methanamine into the tetrahydropyran-di-amine.

2. The process according to claim 1, comprising the preparation of a tetrahydropyran-di-amine represented by the formula: 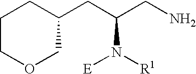
wherein the process comprises the steps of:
1) converting a chloro-pentenol having the formula: 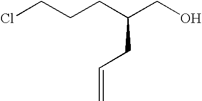
into a propenyl tetrahydropyran having the formula: 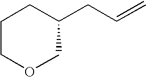
2) converting the propenyl tetrahydropyran into a tetrahydropyran ethylidene methanamine having the formula: 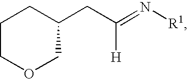
and
3) converting the tetrahydropyran ethylidene methanamine into a cyano-tetrahydropyran-amine having the formula: 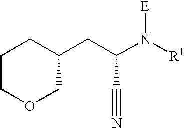
4) converting the tetrahydropyran ethylidene methanamine into the tetrahydropyran-di-amine.

3. The process according to claim 1, further comprising the preparation of a chloro-pentenol having the formula: 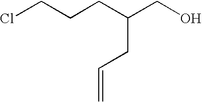
comprising the steps of:
1) treating pseudoephedrine with 5-chloropentanoyl chloride to form a pentanamide having the formula: 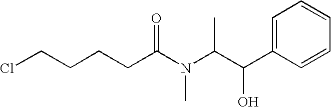
2) converting the pentanamide to a pentenamide having the formula: 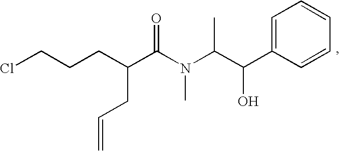
and
3) converting the pentenamide into the chloro-pentenol.

4. The process according to claim 1, further comprising a process for the preparation of an R-chloro-pentenol having the formula: 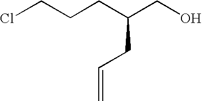
comprising the steps of:
1) treating S,S-pseudoephedrine with 5-chloropentanoyl chloride to form an R,S,S-pentanamide having the formula: 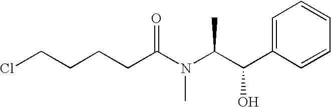
2) converting the R,S,S-pentanamide to an R,S,S-pentenamide having the formula: 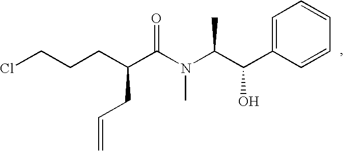
and
3) converting the R,S,S-pentenamide into the R-chloro-pentenol.

5. The process according to claim 1, further comprising a process for the preparation of the tetrahydropyran ethylidene methanamine having the formula: 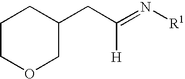
comprising the steps of:
1) converting a chloro-pentenol having the formula: 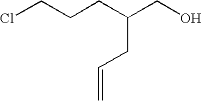
into a propenyl tetrahydropyran having the formula: 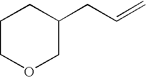
2) converting the propenyl tetrahydropyran into a tetrahydropyran acetaldehyde having the formula: 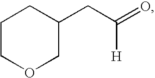
and
3) converting the tetrahydropyran acetaldehyde into the tetrahydropyran ethylidene methanamine.

6. The process according to any one of claims 1-5, further comprising a process for the preparation of the an R-tetrahydropyran ethylidene methanamine having the formula: 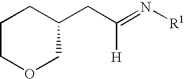
comprising the steps of:
1) converting an R-chloro-pentenol having the formula: 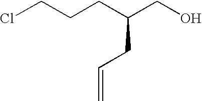
into an R-propenyl tetrahydropyran having the formula: 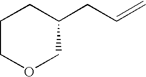
2) converting the R-propenyl tetrahydropyran into an R-tetrahydropyran acetaldehyde having the formula: 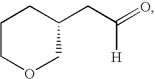
and
3) converting the R-tetrahydropyran acetaldehyde into the R-tetrahydropyran ethylidene methanamine.
